(19) United States
(12) Patent Application Publication
(10) Pub. No.: US 2024/0188800 A1
(43) Pub. Date: Jun. 13, 2024

(54) VALVE FOR A MEDICAL INSTRUMENT, AND MEDICAL INSTRUMENT

(71) Applicant: KARL STORZ SE & Co. KG, Tuttlingen (DE)

(72) Inventors: Alexander FUCHS, Tuttlingen (DE); Tobias LOTTES, Helmbrechts (DE)

(73) Assignee: KARL STORZ SE & Co. KG, Tuttlingen (DE)

(21) Appl. No.: 18/554,780

(22) PCT Filed: Apr. 11, 2022
(86) PCT No.: PCT/EP2022/059653

§ 371 Date: Oct. 10, 2023

(30) Foreign Application Priority Data
Apr. 12, 2021 (DE) 10 2021 109 024.5

(51) Int. Cl. A61B 1/00

(52) U.S. Cl. CPC A61B 1/00068; A61B 1/00094

(57) ABSTRACT

A valve according to the invention, in particular a suction valve, for a medical instrument comprises a valve housing with a cavity which extends in a longitudinal direction and into which, at a longitudinal side of the cavity, a first outlet and a second outlet open in a manner offset relative to each other in the longitudinal direction, and a valve piston which is displaceable in the cavity in the longitudinal direction between an open position and a closed position of the valve and which has a recess through which, in the open position, the first outlet and the second outlet are fluidically connected to each other, wherein the valve piston has a first seal, which circumferentially surrounds the recess. The invention also relates to a medical instrument, in particular a medical endoscope or a medical endoscopic instrument, having such a valve.

### CROSS-REFERENCE TO RELATED APPLICATIONS

This application is the U.S. national stage of PCT/EP2022/059653 filed on Apr. 11, 2022, which claims priority of German Patent Application No. 10 2021 109 024.5 filed on Apr. 12, 2021, the contents of which are incorporated herein.

### TECHNICAL FIELD

The present disclosure relates to a valve, in particular a suction valve, for a medical instrument and to a medical instrument having such a valve.

### BACKGROUND

When performing an endoscopic procedure, it is often necessary to drain fluid from an internal body cavity. For example, it may be necessary to aspirate an irrigation fluid used to irrigate a surgical region located in the body-internal cavity from said surgical region. To control the aspiration, the practice of arranging a manually actuatable suction valve on a handle of the endoscope used to perform the endoscopic procedure is known. A proximal portion of a suction line is connected to the valve and can be used to connect a suction pump, which provides the negative pressure required for aspiration, to the endoscope. Furthermore, a distal portion of the insufflation line is connected to the valve and guided through the shaft of the endoscope to the distal end of the latter. Actuation of the suction valve allows a fluid connection between the distal and proximal portions of the suction line to be established or interrupted, in order to control the aspiration in this way.

EP 3 417 906 A1 discloses a medical pushbutton valve having a valve housing, a flow channel which is formed in the valve housing, and a valve piston which is displaceably arranged in the valve housing between a position releasing the flow channel and a position closing the flow channel. Here, when viewed in the direction of the longitudinal axis of the valve housing, two closure bodies are formed at a distance from one another on the valve piston and interact with corresponding valve seats. A connecting piece for a line leading to the patient is arranged at the distal end of the valve housing. A suction line for applying the suction power coming from an external suction source opens laterally into the valve housing at an acute angle.

However, the installation space available in a handle of an endoscope or any other medical instrument is not always sufficient for a valve with such an arrangement of the connections on the valve housing. Further, the applied negative pressure can give rise to a force which acts on the valve piston in or against an actuation direction, whereby the operability of the suction valve may be impaired. Finally, the manufacture of the valve is relatively complex and potentially error-prone, with the result that the valve is not optimal for use in a disposable instrument.

US 2017/0055942 A1 has disclosed an adjustment device for an ultrasound examination, comprising a change unit for changing a suction force and a damper for suppressing changes in the suction force. The change unit comprises a cylinder having a first and a second portion, which communicate with one end portion and another end portion of a channel which leads to a contact portion, and a piston which is displaceable in the cylinder to change a communication state between the first and the second portion. Actuating the piston allows a contact state to be changed, in particular by periodic switching of the contact state, in order to obtain a good elastographic image of a living tissue.

### SUMMARY

It is an object of the present disclosure to provide a valve, in particular a suction valve, for a medical instrument, with the aforementioned disadvantages being avoided as far as possible. In particular, it is an object of the disclosure to specify a suction valve which has a particularly compact arrangement and/or which has improved operability and/or which can be produced particularly cost-effectively. Furthermore, it is an object of the present disclosure to provide a medical instrument having such a valve.

This object is achieved by a valve as claimed in claim 1 and by a medical instrument as claimed in claim 14.

Advantageous developments of the disclosure arise from the dependent claims.

A valve according to the disclosure for a medical instrument serves to control a fluid flow while the medical instrument is used. The medical instrument can be, for example, a medical endoscope or an endoscopic instrument, wherein the valve can serve to control a fluid flow during an endoscopic procedure performed using the endoscope or the endoscopic instrument. The valve is designed in particular as a suction valve and can consequently serve to control a suction flow and hence to control the aspiration of a fluid, for example an irrigation fluid and/or a bodily fluid during a medical procedure. Then again, the valve can also be used as an insufflation and/or irrigation valve and can therefore be suitable for controlling insufflation or an irrigation process during a medical procedure.

The valve according to the disclosure comprises a valve housing with a cavity which extends in a longitudinal direction and into which, on a longitudinal side of the cavity, a first outlet and a second outlet open in a manner offset from one another in the longitudinal direction. The cavity can be substantially columnar, preferably in the form of a column with a non-circular cross section. The cavity can be terminated at one end by a base, and hence be designed as a blind hole, or can form a cutout that extends through the valve housing. In particular, the first and the second outlet extend at least approximately in a longitudinal plane, which includes a longitudinal axis or the longitudinal direction of the cavity, and are directed toward the same side starting from the longitudinal axis. The first and the second outlet preferably extend across the longitudinal axis: in particular, the first and the second outlet can be directed approximately parallel to one another and approximately perpendicular to the longitudinal direction. The first and the second outlet can each be formed by a connecting piece, to which a respective fluid line can be connected in order to use the valve to control a fluid flow: the first and the second outlet are therefore also referred to herein as the first and the second fluid outlet, respectively. For use as a suction valve, the fluid lines can be designed as portions of a suction line, with for example a proximal portion of the suction line for connection to a suction pump being able to be connected to one of the two fluid outlets and a distal portion of the suction line guided within the medical instrument being able to be connected to the other of the two fluid outlets. The valve housing preferably only has two fluid outlets.

Further, the valve comprises a valve piston which is displaceable in the cavity in the longitudinal direction between an open position, in which there is a fluid connection between the first and the second outlet, and a closed position, in which the fluid connection between the first and the second outlet is interrupted. In this context, “fluid connection” is understood to mean a connection that enables the flow of a fluid, i.e. a gas or a liquid. When the valve is used as a suction valve during a medical procedure, this may in particular be a fluid, which for example can be an irrigation or a bodily fluid possibly also containing tissue parts, or else air or an insufflation gas, for example. The valve piston is in particular manually displaceable, for which purpose the valve piston can be connected to a pushbutton that is actuatable by a finger. The valve piston, which can also be referred to as a tappet or as a plunger, is guided in the cavity in the longitudinal direction, for which purpose the cavity and the valve piston can be embodied with cross sections that are correspondingly matched to one another. The valve piston can be pretensioned by spring force, in particular into the closed position, with the result that, when the valve piston is not actuated, the valve is closed and can be opened by pushing in the pushbutton against the spring force for example.

The valve piston has a recess via which the first and the second outlet are fluidically connected to one another in the open position. For this purpose, the recess is designed and arranged for corresponding interaction with the first and the second outlet. In particular, the recess is a lateral recess in the valve piston, which is arranged on that longitudinal side of the valve piston that faces the first and the second outlet and which extends into the valve piston from said longitudinal side, the recess in this longitudinal side having an opening with a sufficient length in the longitudinal direction to establish the fluid connection between the first and the second outlet. In particular, the recess is designed as a bowl-shaped or trough-shaped depression in the longitudinal side of the valve piston. A fluid flow can flow from the first outlet through the recess to the second outlet, or vice versa, when the valve is in the open position. In this case, the fluid flow within the recess is substantially in the longitudinal direction of the valve piston. Provided that the first and the second outlet open into the cavity parallel to one other and perpendicular to the longitudinal direction, the flow direction of the fluid flow changes by 90° during the transition from the first outlet into the recess and again by 90° during the transition from the recess to the second outlet, and thus in total by 180°.

According to the disclosure, the valve piston has a first seal which surrounds the recess all around. In particular, the first seal is arranged on or adjacent to an edge of the recess or the opening in the recess on the longitudinal side of the valve piston and thus surrounds the recess. In a particularly preferred manner, the first seal surrounds the recess without gaps. To seal the recess and hence the flow path of the fluid flow able to flow through the recess in the open position of the valve, the first seal interacts with a corresponding sealing surface of the valve housing. The sealing surface in particular encloses the mouths of the first and the second outlet in the cavity, the sealing surface preferably surrounding the mouths on all sides, and the first seal also surrounds the mouths of the first and the second outlet in the open position. Preferably, the sealing surface surrounds each of the two mouths on all sides. In particular, in the open position, the first seal seals both outlets, which are connected to one another, against the cavity of the valve housing: in the closed position, one of the two outlets can be in fluid communication with the recess and is also sealed by the first seal.

The fact that, according to the disclosure, a first seal is provided, which is assigned to the valve piston and surrounds the recess all round, in particular in full, renders it possible to easily and reliably establish or interrupt a fluid connection between the first and the second fluid outlet. Particularly when the valve is used as a suction valve, a fluid connection with a sufficient cross section and sufficient tightness is achievable, the latter enabling an aspiration of fluids from a cavity or surgical region located within a human or animal body, wherein the fluids have different viscosities and may also carry along separated parts of tissue. Furthermore, guidance of the valve piston in the cavity of the valve housing can be improved by the first seal, which is arranged on the valve piston. Furthermore, the fact that the valve piston has the first seal enables more cost-effective and more process-reliable production.

The valve according to the disclosure can, for example, be designed as a disposable part, be destined for one-time use (single use), and consist of materials that are advantageous for one-time use. Thus, in this case, at least the valve housing and the valve piston with the first seal may consist of cost-effective plastic materials, which allow a simple production, for example by injection molding, but need not be suitable for cleaning and reprocessing. The valve according to the disclosure can be provided for installation in a medical instrument, for example in a handle of an endoscope, wherein the medical instrument can be in particular a disposable instrument or a disposable endoscope. Then again, the valve can also be intended for separate use or for use in its own control unit.

Furthermore, provision can be made for the valve piston to be designed with drafts, whereby a production as an injection-molded part is likewise simplified. This makes particularly cost-effective manufacture possible, which is particularly advantageous in the case of a design as a disposable part. The valve housing, the pushbutton and optionally further components of the valve can likewise be produced as injection-molded parts.

The valve piston may have further positions in addition to the open position and the closed position, for example one or more intermediate positions between the closed position and the open position, in which a cross section available for the fluid flow can be restricted for metering the fluid flow. Preferably, the recess is designed and, in the open position, arranged in such a way that the mouths of the first and the second fluid outlet into the cavity each open into the recess with their entire cross section or lie opposite an opening in the recess and interact therewith to establish the fluid connection. Furthermore, in this case, provision can be made for the valve piston to be able to be brought into one or more intermediate positions, with only part of the cross section of the mouth of the first and/or the second outlet merging into the recess or lying opposite the opening of the recess. This can result in a corresponding reduction in the cross section in the transition area and thereby bring about a metering of the fluid flow or, when used as a suction valve, a change in the suction effect.

According to a preferred embodiment of the disclosure, the valve piston has a closure region which is arranged on the same longitudinal side of the valve piston as the recess and offset in the longitudinal direction from the recess, and the recess and the closure region are designed and arranged in such a way that, in the closed position of the valve piston, the first outlet is connected to the recess but the second outlet is sealed by the closure region. In a particularly preferred manner, the valve piston can have a second seal which surrounds the closure region all round. To seal the second outlet in the closed position, the second seal interacts with a sealing surface which is part of the valve housing and which encloses at least the mouth of the second outlet into the cavity of the valve housing. The sealing surface of the valve housing is preferably designed to interact with the first and the second seal in such a way that, in the closed position, the first seal surrounds the mouth of the first outlet and seals the latter tightly against the cavity of the valve housing and, at the same time, the second seal surrounds the mouth of the second outlet and also seals the latter tightly against the cavity, while in the open position the first seal surrounds the mouths of the first and the second outlet and seals these jointly against the cavity. The first and the second seal may have a common portion: in particular, the recess of the valve piston and the closure region can be arranged adjacent to one another, the edge of the recess adjacent to the closure region having a portion of the first seal which is also a portion of the second seal at the same time. Advantageously, the recess can be located closer to that end of the valve piston at which an actuating force for displacing the valve piston can be exerted using a pushbutton, and the closure region can be located away from this end: accordingly, the first outlet is then closer to the pushbutton than the second outlet. This makes it possible to open the valve in a particularly simple manner by actuating the pushbutton against a spring force.

According to an advantageous aspect of the disclosure, the sealing surface of the valve housing, which interacts for sealing purposes with the first seal, is a substantially flat surface. Preferably, a sealing surface with which the second seal interacts for sealing purposes is also a substantially flat surface. Particularly preferably, the two aforementioned sealing surfaces form a common, at least substantially flat surface. According to this aspect of the disclosure, a cross section of the cavity of the valve housing is therefore not circular but delimited by an at least approximately flat surface, at least on a longitudinal side into which the first and the second outlet open. The fact that the sealing surface of the valve housing is an at least substantially planar surface not only enables an improved sealing effect of the first or the second seal, but also enables, at the same time, a guidance of the valve piston in the cavity in a manner secured against rotation about the longitudinal axis.

The cavity preferably has an approximately rectangular cross section. The valve piston too may have an at least approximately rectangular cross section. This makes it possible to obtain further improved sealing and particularly precise, rotationally fixed guidance of the valve piston, and hence improved operability of the valve.

According to an embodiment of the disclosure, the cavity of the valve housing can be designed to narrow in a wedge shape toward the closed position, the valve piston preferably likewise being designed to have a corresponding wedge shape. In particular, the closed position of the valve piston is an end position of the valve piston in the direction of the pushbutton, and, by pressing the pushbutton into the valve housing, the valve piston can be displaced into the open position, wherein the valve piston can be preloaded into the closed position, for example by a spring force. In this case, according to this embodiment, the cavity and likewise the valve piston narrow in a wedge shape toward the pushbutton. What this can achieve is that, in the region of the closed position, there is a tight boundary as a result of the first and possibly the second seal, whereas the valve piston only rests loosely against a wall of the cavity in all other positions and hence there is only little friction. When the valve is used as a suction valve, a negative pressure applied to the first or second outlet can be sufficient here for drawing the valve piston toward a corresponding sealing surface of the housing, and hence sufficient for sealing purposes.

According to a further advantageous aspect of the disclosure, the first seal is formed in one piece, in particular in one part, with the valve piston. This means that the first seal is part of the valve piston and forms a single component with the latter: in particular, the first seal is produced with the valve piston as a single component. For example, the valve piston can be produced with the first seal as an injection-molded part, with the valve piston and the first seal consisting of the same material. In a likewise advantageous manner, the second seal can be formed in one piece with the valve piston. Preferably, the first and the second seal are formed in one piece with the valve piston. This reduces the number of components of the valve, in particular making it possible to produce the valve piston including the first and optionally the second seal as a single part, for instance as an injection-molded part. As a result, the number of manufacturing steps can be reduced, the process reliability can be increased and cost-effective manufacture can be made possible.

Advantageously, at least the first seal can be formed at least in portions by a sealing lip, which can have, for example, a semicircular, a V-shaped, or a wedge-shaped cross section. If the valve piston, including the first seal, has an excess in relation to a cross section of the cavity, then the first seal is compressed, whereby a high normal force and therefore a high frictional force may arise in the case of a surface seal. In the case of a sealing lip, especially with a semicircular, V-shaped, or wedge-shaped cross-sectional profile, there is in each case only a substantially linear contact with the sealing surface, with the result that, in the event of an excess, only a smaller volume of the seal is compressed, and so only a smaller normal force arises, the latter however increasing more pronouncedly with increasing excess. As a result of the seal being designed as a sealing lip, especially with a semicircular, V-shaped, or wedge-shaped profile, it is possible to obtain, firstly, a sufficient sealing effect and, secondly, a reduction in the friction between the valve piston and the valve housing when the valve is actuated. Alternatively, the first seal can be designed as a flat seal or surface seal, which enables a flat, particularly secure seal.

The second seal can likewise advantageously be designed at least in portions as a sealing lip with a, for example semicircular, V-shaped, or wedge-shaped, cross-sectional profile. In a particularly preferred manner, the first and the second seal, optionally also a common portion of the first and the second seal, are designed as a sealing lip with the same cross-sectional profile. This allows the operability of the valve to be further improved. Alternatively, the second seal, in particular the first and the second seal and possibly a common section, can be designed as a flat seal or surface seal, thereby enabling a flat seal.

In a particularly advantageous manner, provision can be made for the valve piston to have a counterholder on a longitudinal side opposite the first seal, the counterholder preferably being arranged opposite the first seal and, in the open position, opposite the first and the second outlet, in each case relative to the longitudinal axis. Provided that the valve piston has a second seal which surrounds the closure region all round, the counterholder is preferably also arranged opposite the closure region and, in the closed position, opposite the second outlet. As a result of the counterholder, it is possible, together with a corresponding dimensional coordination of the cross sections of the valve piston and the cavity, to generate a counterforce and a pretension of the first and the second seal in the direction of the respective sealing surfaces. As a result of the counterholder, which also acts as a counterbearing, it is thus possible to obtain an improved contact of the first and/or the second seal on a corresponding sealing surface on the longitudinal side of the cavity and consequently an increased sealing effect and also improved guidance of the valve piston and reduced friction.

The counterholder can comprise at least one counterholder element, which can be designed at least in portions in a manner corresponding to the first seal and/or the second seal. In particular, the counterholder can comprise two strip-shaped counterholder elements which extend in the longitudinal direction and are spaced apart from one another across the longitudinal direction and which ensure secure, play-free guidance of the valve piston in the cavity.

The at least one counterholder can also advantageously be designed, at least in portions, as an elevation with a, for example semicircular, V-shaped, or wedge-shaped, cross-sectional profile: to this end, the latter need not have a sealing effect but only needs to serve the generation of the counterforce. In a particularly preferred manner, the counterholder is designed as an elevation with an identical cross-sectional profile to a sealing lip which forms the first and/or the second seal, in particular as two longitudinally extending portions of such a sealing lip which are spaced apart across the longitudinal direction. This makes it possible to reduce friction and further improve the operability of the valve.

Preferably, the counterholder or the at least one counterholder element is designed in one piece with the valve piston. In a particularly preferred manner, the first seal and the counterholder or the at least one counterholder element and optionally the second seal are formed in one piece with the valve piston. This makes it possible to produce the valve piston including the counterholder and in particular including the first and the second seal as a single part, for instance as an injection-molded part, whereby particularly cost-effective production is made possible.

The valve piston preferably has an at least substantially uniform wall thickness. For this purpose, provision can be made, in particular, for a further recess to be provided in the closure region or on a side of the valve piston opposite the closure region. The further recess does not impair the sealing effect of the closure region but allows the valve piston to be designed with a substantially uniform wall thickness. This allows the production to be improved further, especially as an injection-molded part.

Advantageously, provision can be made for the valve piston to be provided with one or more reinforcing ribs, in particular with one or more reinforcing ribs which extend across the longitudinal direction and are arranged in the recess and/or on the side of the valve piston opposite the recess. Provided that one or more reinforcing ribs are arranged in the recess which serves as a fluid connection between the first and the second outlet, said reinforcing ribs can be dimensioned in such a way that a cross-sectional area of the recess available for the fluid flow is no smaller than that of the first and the second outlet. Provided that one or more transverse ribs are arranged on the opposite side of the recess, these preferably do not protrude beyond the at least one counterholder element. As a result, a deformation of the valve piston caused by an actuation force and/or frictional forces during actuation and/or as a result of pressure forces can be minimized.

According to an embodiment of the disclosure, at least one end face of the valve piston has a rounded-off form and in particular forms a convexly curved end surface. The at least one end face is preferably the one at which an actuating force is exerted on the valve piston for the purpose of displacing the latter in the longitudinal direction: both end faces are particularly preferably shaped in this way. In particular, the at least one end face is designed such that, in a longitudinal section of the valve piston in a plane to which the two outlets are approximately perpendicular, the end face is approximately semicircular with an outward curvature: both end faces are preferably designed in this way. The recess and optionally the further recess can have correspondingly curved side walls, with the result that the valve piston has a uniform wall thickness. This can likewise reduce a deformation of the valve piston caused by the actuating force.

Advantageously, the valve housing can be made of a stiffer material than the valve piston. By way of example, the valve housing can consist of polycarbonate or another thermoplastic material such as polyetheretherketone (PEEK), polyphenylene sulfone (PPSU), or acrylonitrile-butadiene-styrene copolymers (ABS), while the valve piston, preferably including at least the first seal and optionally the second seal and/or the counterholder, can consist of polyethylene (PE) or else, for instance, of thermoplastic elastomers (TPE), polypropylene (PP), or thermoplastic polyurethane (TPU). This renders lower friction and a good sealing effect achievable.

A medical instrument according to the disclosure, in particular a medical endoscopic instrument or a medical endoscope, comprises a valve with the above-described design. The valve can for example be installed in a handle of the instrument or endoscope and be arranged in such a way that a user can hold the handle with one hand and operate the valve with one finger of the hand by pushing in the pushbutton. The medical instrument or endoscope can in particular be designed as a disposable instrument or disposable endoscope.

It is understood that the features mentioned above and the features yet to be explained below are applicable not only in the respectively specified combination but also in other combinations or on their own, without departing from the scope of the present disclosure.

Further aspects of the disclosure emerge from the following description of preferred exemplary embodiments and the attached drawing.

### BRIEF DESCRIPTION OF DRAWINGS

FIG. 1 shows an endoscope having a valve according to an exemplary embodiment of the disclosure:

FIG. 2 shows the handle of the endoscope according to FIG. 1 in an opened view:

FIG. 3 shows the proximal end region of the handle according to FIG. 2 in an enlarged sectional view:

FIGS. 4a and 4b show the valve according to FIGS. 1 to 3 in an overall view and an exploded view:

FIGS. 5a to 5c show a schematic longitudinal section through the valve according to FIGS. 1 to 4b in three different positions of the valve piston;

FIGS. 6a and 6b show the valve piston of the valve according to a first embodiment:

FIGS. 7a and 7b show the valve piston according to a second embodiment:

FIG. 8 shows the valve piston according to a third embodiment:

FIGS. 9a and 9b show the valve piston according to a fourth embodiment:

FIGS. 10a and 10b show the valve piston according to a fifth embodiment:

FIGS. 11a and 11b show the valve piston according to a sixth embodiment:

FIGS. 12a to 12c show variants of the fourth to sixth embodiments of the valve piston;

FIG. 13 shows the valve piston according to a seventh embodiment;

FIGS. 14a and 14b show a schematic longitudinal section through the valve according to a further exemplary embodiment in two different positions of the valve piston.

### DETAILED DESCRIPTION OF PREFERRED EMBODIMENTS

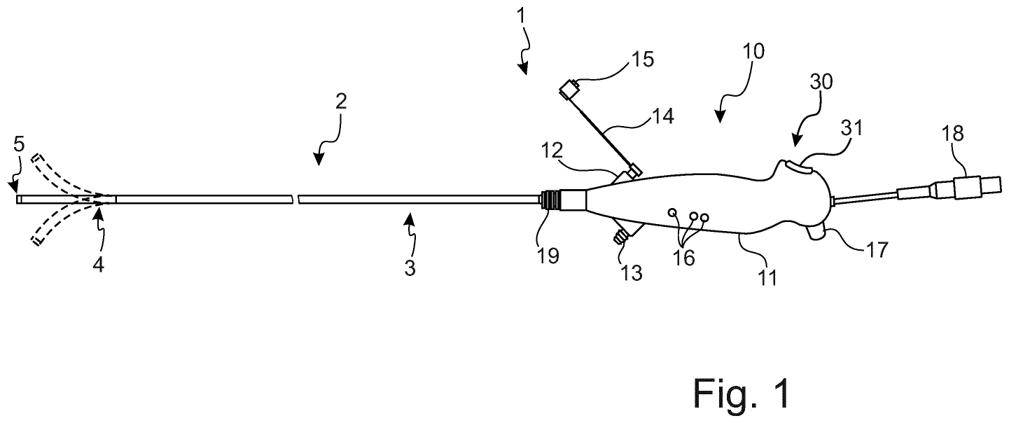

FIG. 1 shows, by way of example, an endoscope having a valve according to the disclosure in an overall view. The endoscope 1 is a flexible endoscope and comprises an elongate flexible shaft 2 which is designed for insertion into a body-internal cavity of a human or animal body. The shaft 2 comprises a first shaft portion 3, with a second shaft section 4 being arranged at the distal end, i.e. the end remote from the user, of said first shaft portion. The first shaft portion 3 is flexible and can, for example, adapt to the shape of a curved access path to a body-internal cavity. As indicated in FIG. 1, the second shaft portion 4 is controllable and can be curved from an elongate to an angled arrangement at least in one plane. A camera head 5 is arranged at the distal end of the second shaft portion 4, which is also referred to as a controllable shaft portion.

At a proximal end, which is to say the end near the user, the shaft 2 is connected to a handle 10, which serves to operate the endoscope 1 and for this purpose has an ergonomically shaped housing 11, on which operating elements and connections for further equipment are arranged. In particular, the housing has an access port 12 for inserting a working instrument into a working channel that extends to the camera head 5, and also a tube connector 13 for connecting a suction or irrigation tube, the access port 12 being closable with a closure cap 15 held on a flexible loop 14. A plurality of operating elements 16 for controlling various functions of the endoscope 1, such as light and zoom, are arranged on the housing 11.

Furthermore, the handle 10 comprises a control mechanism for bending the controllable second shaft portion 4, which can be actuated by means of a lever 17. Furthermore, an electrical connection 18 is connected to the housing 11 via a cable, which is longer than depicted and which serves for connecting the endoscope 11 to a supply and evaluation device, not shown, in order to supply electrical and electronic components of the endoscope with electrical energy and to display and process a recorded endoscopic image. A plurality of retaining lips 19 are arranged in the distal end region of the handle 10, in which the first shaft portion 3 is connected to the housing 11, in order to be able to seal the endoscope 1 when used in an insertion instrument.

Further, the handle 10 comprises a valve 30 according to an exemplary embodiment of the disclosure, which is described in more detail hereinbelow. In the exemplary embodiments described, the valve 30 is a suction valve which is operable by a pushbutton 31 to control the aspiration of irrigation fluid and/or bodily fluid from a body-internal cavity into which the shaft 2 has been inserted.

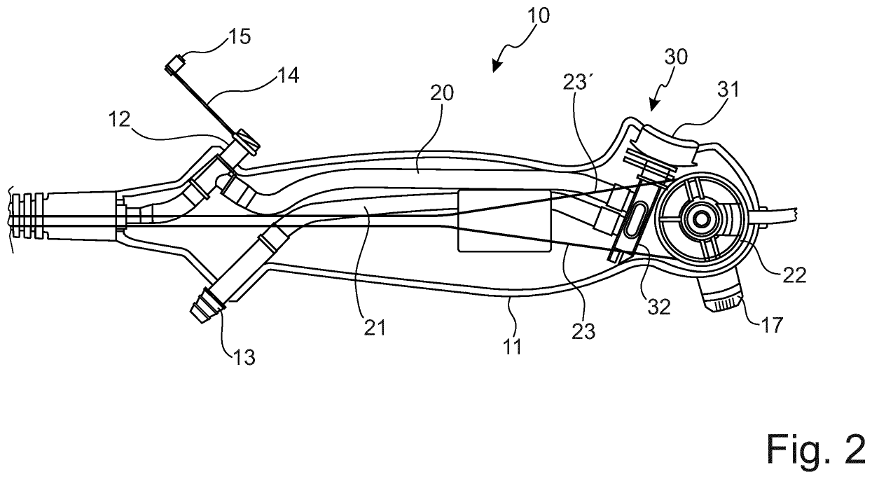

As shown in FIG. 2, a tube 20 laid within the housing 11 branches off from the access port 12 and is used to connect the working channel that extends within the shaft 2 to a first outlet of the valve 30. A second outlet of the valve 30 is connected to the tube connector 13 by means of a further tube 21 which is likewise laid within the housing 11.

Further components are accommodated in the housing 11. Thus, a control wheel 22 is rotatably mounted in the proximal end region of the handle 10 and used to guide two pull wires 23, 23′ which act counter to one another and extend through the shaft 2 into the controllable second shaft portion 4: the pull wires 23, 23′ can also be designed as portions of a continuous wire guided over the control wheel 22. The control wheel is rotatable manually by means of the lever 17, whereby the second shaft portion 4 can be actively bent (see FIG. 1).

To aspirate irrigation fluid and/or bodily fluid from a body-internal cavity, the access port 12 is closed by means of the closure cap 15 and a suction tube, which establishes the connection to an external suction pump, is connected to the tube connector 13. The tube 20 connected to the working channel thus represents a distal portion of a suction line and the further tube 21 connected to the tube connector 13 represents a proximal portion of the suction line, the valve 30 being connected between the proximal and the distal portion of the suction line. A fluid connection between the proximal and the distal portion of the suction line can be established or interrupted by means of the valve 30, and the aspiration can thus be controlled.

The valve 30 is arranged in the proximal end region of the handle 10. The housing 11 of the handle 10 is ergonomically designed in such a way that the handle 10 can be held by a user with one hand and, in the process, the pushbutton 31 of the valve 30 is actuatable using a finger, for example the index finger of the same hand. At the same time, the lever 17 for controlling the angle of the controllable second shaft portion 4 can for instance be operated by the thumb of the same hand. In accordance with the installation space available within the housing 11 in the proximal end region and the ergonomic arrangement of the pushbutton 31, the two tubes 20, 21 are connected to the valve 30 on the same side of the valve, in a manner running parallel to one another.

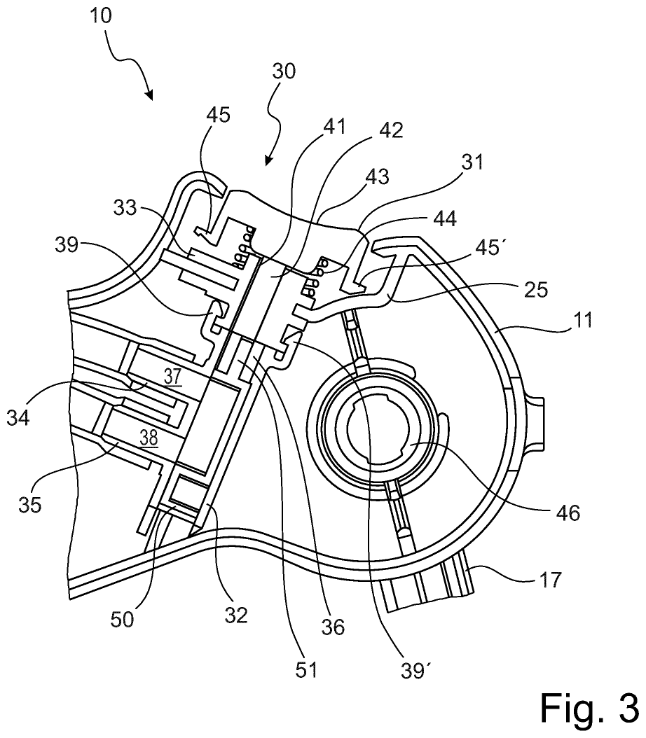

In FIG. 3, the proximal end region of the handle 10 is shown in a longitudinal section. The valve 30 comprises a valve housing 32, a holding part 33, the pushbutton 31, and a first and a second connecting piece 34, 35. The valve housing 32 is designed in the form of an elongate shaft with an approximately rectangular cross section, which encloses a continuous cavity 36 which is approximately rectangular in cross section. A valve piston 50, which is likewise approximately rectangular in cross section, is guided in a longitudinally displaceable manner into the cavity 36. The first and the second connecting piece 34, 35 are fixed to a longitudinal side of the shaft-shaped valve housing 32 in a manner spaced apart in the longitudinal direction from one another and directed parallel to one another, and each of these connecting pieces encloses a continuous interior space which forms a first outlet 37 and a second outlet 38 of the cavity 36 of the valve housing 32. The connecting pieces 34, 35 or the outlets 37, 38 each have a cylindrical or conical frustum shape, and hence each open into the cavity 36 with a circular mouth.

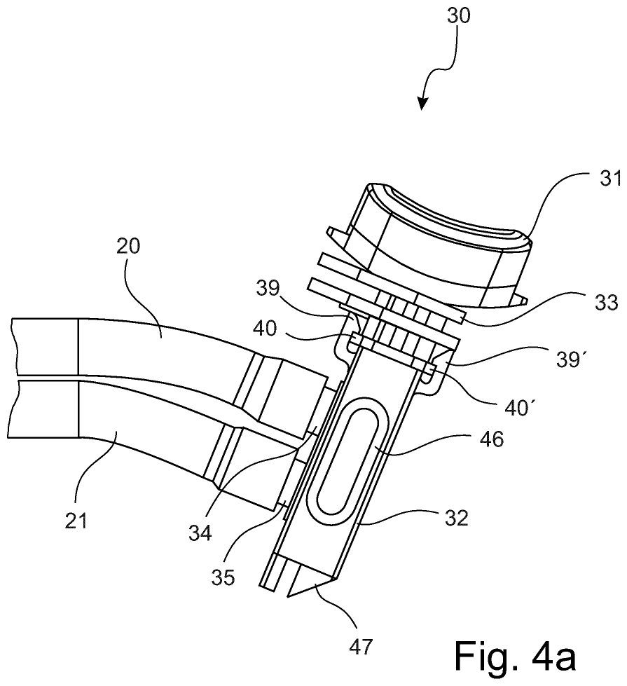

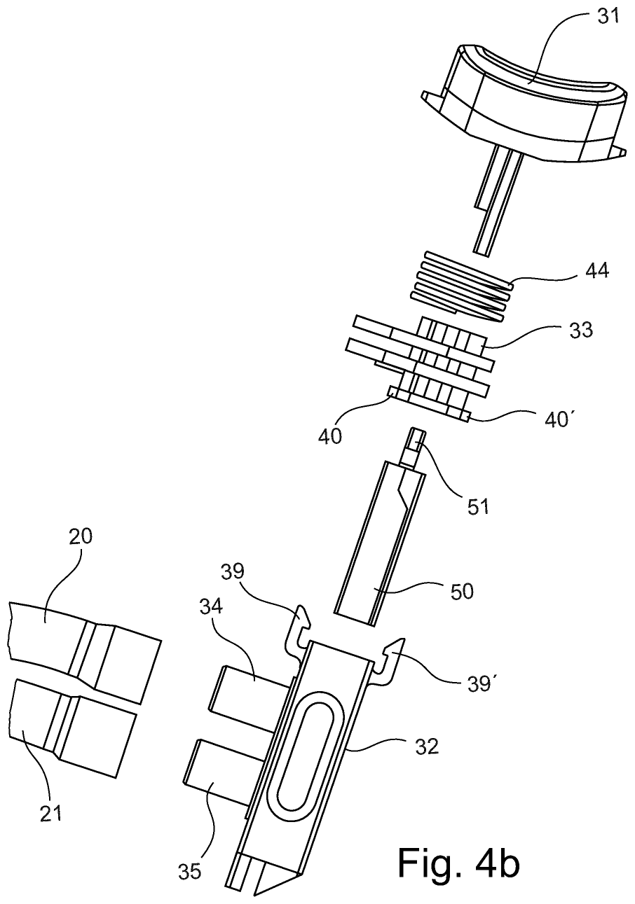

At its head end facing the pushbutton 31, the valve housing 32 is connected to the holding part 33 via two locking hooks 39, 39′ which engage behind corresponding locking lugs 40, 40′ of said holding part 33 (see also FIGS. 4a, 4b). The holding part 33 is inserted into a recess in an intermediate wall 25 of the housing 11 of the handle 10, whereby the valve 30 is held in the interior of the handle 10. An extension 42 of the pushbutton 31 is, in longitudinally displaceable fashion, guided through a cavity 41 of the holding part 33 which adjoins the cavity 36 of the valve housing 32 in the longitudinal direction. A connecting portion 51 of the valve piston 50 has been hooked into the extension 42, with the result that the valve piston in the cavity 36 can be adjusted in the longitudinal direction by actuating the pushbutton 31.

The outer surface of the pushbutton 31 is designed as an ergonomic recessed grip 43. A compression spring 44 is arranged between the holding part 33 and an inner side of the pushbutton 31 and used to preload the pushbutton 31 into a position away from the valve housing 32. By manually exerting a compressive force on the pushbutton 31, the latter can be pressed into the housing 11 and thereby brought into the position shown in FIG. 3, together with the valve piston 50. In this case, an adjustment path of the pushbutton and hence of the valve piston 50 is limited by stop lugs 45, 45′ which act on both sides in conjunction with the housing 11.

Further, a drive shaft is also evident from FIG. 3, on which the control wheel, not shown in FIG. 3, is secured in a rotationally fixed manner and which can be driven by the lever 17.

As shown by the overall view and the exploded view of the valve 30 in FIGS. 4a and 4b, the valve 30 comprises only a small number of individual parts, from which it can be assembled in a few simple assembly steps. For this purpose, the compression spring 44 is inserted between the holding part 33 and the pushbutton 31 and the extension 42 of the pushbutton 31 is guided through the holding part 33, with the connecting portion 51 of the valve piston 50 also being hooked into the extension 42. The valve piston 50 is thereupon inserted into the cavity of the valve housing 32, with the locking hooks 39, 39′ engaging behind the locking lugs 40, 40′, and the tubes 20, 21 being pulled over the connecting pieces 34, 35. As is likewise evident from FIGS. 4a and 4b, the valve housing 32 has a reinforcing rib 46 on the outer side and, at its end distant from the pushbutton 31, a support portion 47 for supporting and further fixing the valve 30 in the housing 11 of the handle 10.

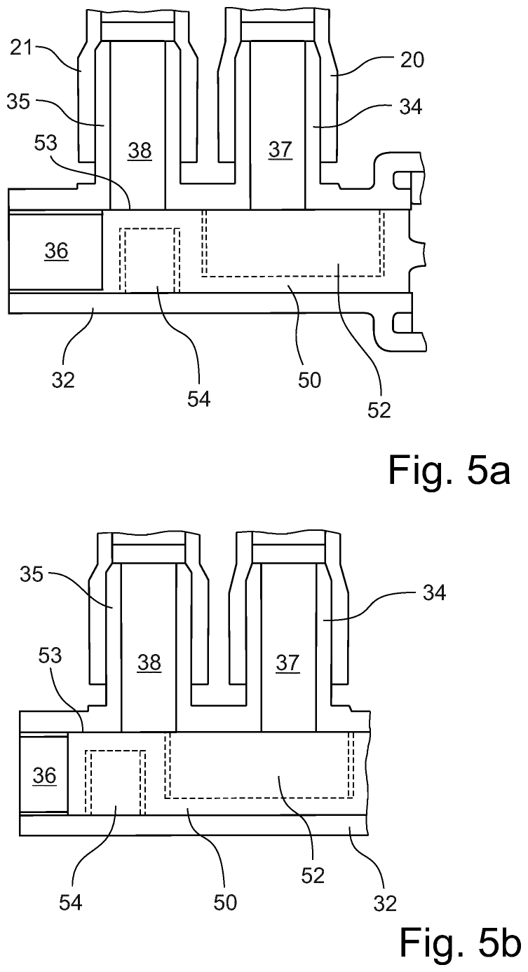

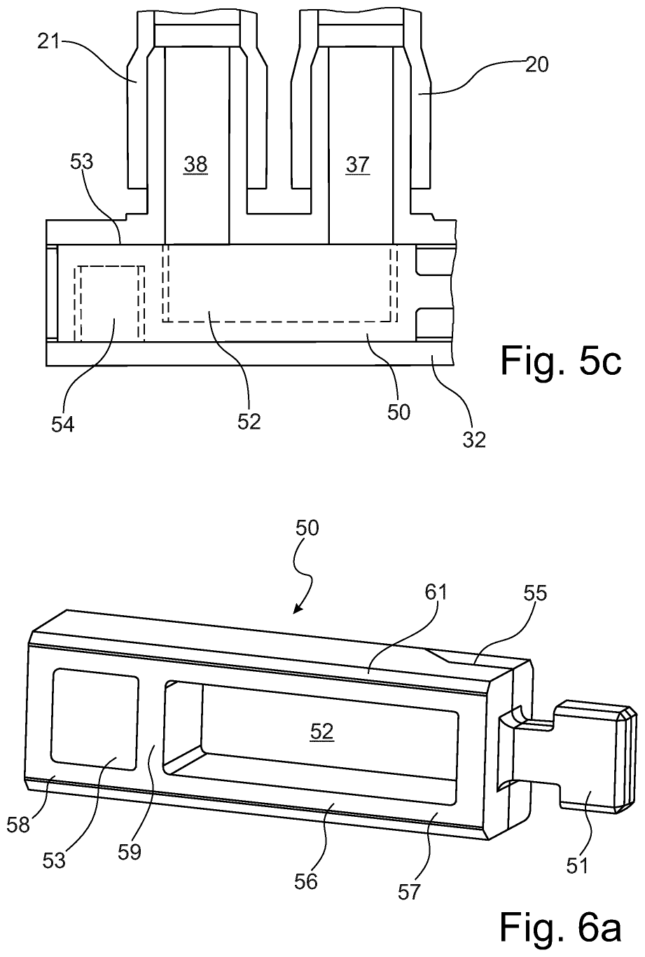

In FIGS. 5a to 5c, the functionality of the valve 30 is shown in a schematic illustration by way of example. The valve piston 50 is mounted in the cavity 36, enclosed by the valve housing 32, so as to be displaceable in the longitudinal direction which runs in the horizontal direction in FIGS. 5a to 5c. As indicated in FIGS. 5a to 5c, the valve piston has a recess 52 which extends into the valve piston from the longitudinal side of the valve piston 50 that faces the first and the second outlet 37, 38. This is bowl-shaped or trough-shaped and is open on the side of the valve piston 50 directed toward the first and the second outlet 37, 38, which is the upper side in the illustration in FIGS. 5a to 5c. In the closed position of the valve piston 50 shown in FIG. 5a, the recess 52 is arranged opposite the first outlet 37 and therefore connected to the latter.

A longitudinal displacement of the valve piston 50 allows the recess 52 to be displaced so far that it is also opposite the second outlet 38 and connected to the latter. While FIG. 5a shows the closed position of the valve piston 50, in which only the first outlet 37 is connected to the recess 52, FIG. 5c shows the open position of the valve 30, in which both outlets 37, 38 are connected to the recess 52, with the result that a fluid connection is established between the first and the second outlet 37, 38. The recess 52 represents a volume of the valve piston 50 through which a flow may pass, with the entire cross section, with which the first and the second outlet 37, 38 open into the cavity 36, being opposite to the recess 52 in each case and hence the first and the second outlet 37, 38 each opening into the recess 52 with their entire cross section.

FIGS. 5a and 5c show the end positions of the valve piston 50. In the intermediate position shown in FIG. 5b, the first outlet 37 is connected to the recess 52 over its entire cross section, but the second outlet 38 is only connected over part of its cross section. An increased flow resistance to a flow through the valve 30 can be obtained as a result. By moving the valve piston 50 into such an intermediate position, which is also referred to as the metering position, metering can take place and, for example, the suction power can be controlled during aspiration.

As shown in FIG. 5a, the second outlet is closed by a closure region 53 of the valve piston 50 in the closed position of the valve or valve piston 50. In the corresponding portion of the valve piston 50, the latter has a further recess 54 which is located on the side facing away from the outlets 37, 38 and which is open towards this side. The further recess 54 allows the valve piston 50 to be designed with a largely uniform wall thickness even in the closure region 53, as is likewise indicated in FIGS. 5a to 5c.

In order to achieve the tightest possible closure of the second outlet 38 in the closed position and, at least in the closed and the open position, a seal against the ingress of fluids into the cavity 36, the valve piston 50 has seals, which are not shown in FIGS. 5a to 5c, on its longitudinal side facing the first and the second outlet 37, 38. A flat sealing surface, with which the seals interact for sealing purposes, is formed on the inner side of the valve housing 32 into which the first and the second outlet 37, 38 open. The remaining longitudinal sides of the cavity 36 are also designed, at least in portions, as flat surfaces.

In the described exemplary embodiments, the valve 30 is designed as a suction valve for controlling aspiration from a body-internal cavity, wherein the flow cross section can be, for example, approximately 7 mm2 at a suction pressure of approx. −0.01 to −0.1 hPa. The travel of the valve piston 50 between the open and the closed position can be approximately 5 mm, for example.

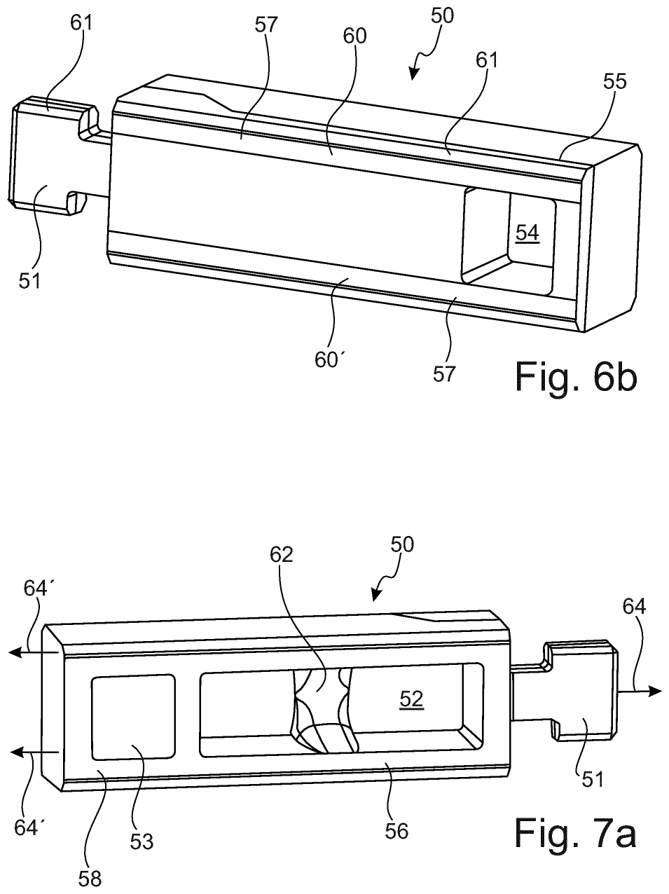

FIGS. 6a to 13 illustrate the valve piston 50 of the above-described valve 30, in each case in enlarged fashion and in accordance with different embodiments of the valve 30. Otherwise, the valve 30 can be designed as described in relation to FIGS. 1 to 5b.

In the first embodiment depicted in FIGS. 6a and 6b in two views from opposite sides, an approximately cuboid recess 52 has been introduced into the valve piston 50 from the side thereof facing the outlets 37, 38, said recess forming a volume, through which flow can pass, serving to establish a fluid connection between the first outlet 37 and the second output 38 in the open position. Adjoining this in the longitudinal direction is the closure region 53, which covers the second outlet 38 in the closed position.

The further recess 54 is introduced into the valve piston 50 from the side facing away from the outlets 37, 38, and it is also approximately cuboid and allows the valve piston 50 to have a largely uniform wall thickness (see FIG. 6b). This simplifies the production as an injection-molded part. FIGS. 6a and 6b also indicate a separation plane 55, at which a corresponding injection mold can be separated after the casting process, with any burrs remaining there not impairing the above-described function of the valve piston 50 for establishing or interrupting a fluid connection between the outlets 37, 38.

A first seal 56 is also indicated in FIGS. 6a and 6b; it surrounds the recess 52 all round and, in the example shown, is designed as a surface seal or flat seal. The first seal 56 is arranged on the edge of the recess 52. The closure region 53 adjoining the recess 52 is surrounded all around by a second seal 58, which is likewise designed as a surface seal and which comprises a common portion 59 with the first seal 56. As described hereinabove, the first and the second seal 56, 58 interact with the sealing surface of the valve housing 32, into which the first and the second outlet 37, 38 open, for sealing in the open and in the closed position. Even in the metering position, the first and the second seal 56, 58, which merge into one another, work together to seal the outlets 37, 38 against the cavity 36. The first and the second seal 56, 58 are each formed in one piece with the valve piston 50 and represent an integral part of the valve piston 50.

In order to achieve sufficient tightness, the first and the second seal 56, 58 are pressed against the sealing surface of the valve housing with pretension. The pretension is achieved, firstly, by dimensional coordination of the valve piston 50 and the cavity 36 in the valve housing 32. In order to reduce the resultant friction and thus facilitate the operation of the valve, the valve piston 50 has a counterholder on its longitudinal side facing away from the outlets 37, 38: in the example shown, this counterholder is formed by two counterholder elements 60, 60′ which extend in the longitudinal direction and, like the seal 56 or 58, are designed as flat elevations. The counterholder serves to generate a counterforce in order to improve the sealing effect of the first and the second seal 56, 58. In addition, when used as a suction valve, an applied negative pressure can improve the sealing effect. The counterholder elements 60, 60′ are also each formed in one piece with the valve piston 50.

As is likewise evident in FIGS. 6a and 6b, the valve piston has a plurality of drafts 61 which facilitate production as an injection-molded part. The first and the second seal 56, 58 and the counterholder elements 60, 60′ are formed in one piece with the valve piston 50, with the result that the latter can be produced as an injection-molded part in a single manufacturing step. To this end, the valve piston 50 is produced for a material that, firstly, is stiff enough to allow the valve piston 50 to be displaced without excessive deformation, but, secondly, is soft enough to ensure the sealing effect of the seals 56, 58. Materials such as PE, TPE, PP, or TPU come into question for this. FIGS. 6a and 6b also show the connecting portion 51, which serves to connect to the extension 42 of the pushbutton 31.

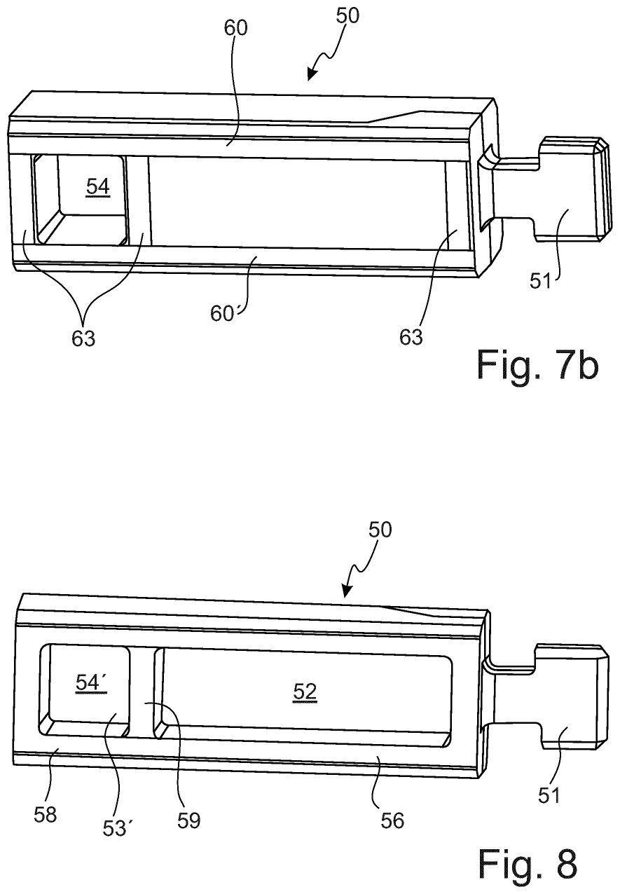

The second embodiment of the valve piston 50 shown in FIGS. 7a and 7b differs from the one described above in that reinforcing ribs 62, 63 are respectively arranged in the recess 52 and on the side of the counterholder. The force introduced centrally with respect to the cross section of the valve piston 50 and acting on the valve piston 50 via the connecting portion 51 leads to deformations of the valve piston 50 on account of the friction of the seals 56, 58 and the counterholder. When the valve 30 is closed, this is a tensile force, which is indicated by the arrow 64 in FIG. 7a: deformations caused thereby are indicated symbolically in FIG. 7a by the arrows 64′. Further, deformations can be caused by the normal force, with which the seals 56, 58 must be pressed against the sealing surfaces of the valve housing 32, and also by the pressure or negative pressure present at the outlets 37, 38. Such deformations may occur in particular if the valve piston 50 consists of a relatively soft material, which is advantageous for the one-piece design with the seals 56, 58 and the counterholder elements 60, 60′, which consist of the same material. These deformations can be minimized by the reinforcing ribs 62, 63, and so the tightness and the operability of the valve 30 are not significantly impaired.

Such reinforcing ribs 62, 63 can also be provided in the other embodiments, described hereinabove and hereinbelow, for the purpose of reducing the deformation of the valve piston 50.

According to a third embodiment, the further recess 54′ can be introduced into the valve piston 50 from the side facing the second outlet 38 in that portion of the valve piston 50 which lies opposite the second outlet 38 in the closed position, as depicted in FIG. 8. In this case, the closure region 53′, which covers and hence closes the second outlet 38 in the closed position, is formed by the back wall and the side walls of the further recess 54′. The sealing effect can be achieved by the circumferential second seal 58, in the same way as described above. Otherwise, the valve piston 50 can be designed as described above.

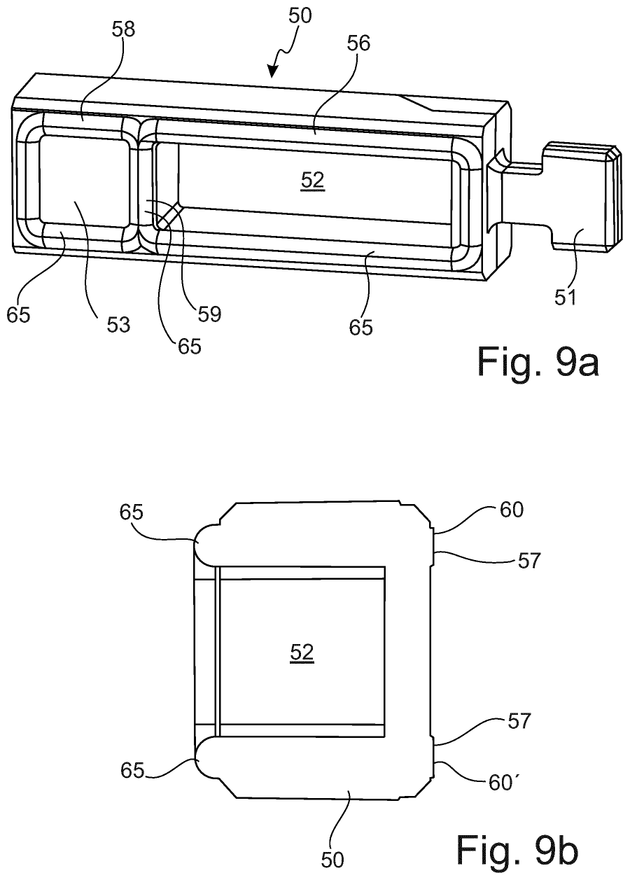

A fourth embodiment of the valve piston 50 is depicted in FIGS. 9a and 9b. Here, the first seal 56 is formed by a sealing lip 65, which has a convex, approximately semicircular cross-sectional profile, as is evident from the cross section through the valve piston 50 shown in FIG. 9b. The second seal 58 is also formed by a sealing lip 65 with an approximately semicircular cross-sectional profile, just like the common portion 59 of the first and the second seal. The semicircular cross-sectional profile brings about a substantially line-shaped contact of the seals 56, 58 with a sealing surface interacting therewith, wherein, if the valve piston 50 including the seals 56, 58 and the counterholder has an excess in comparison with the cross section of the cavity 36, this gives rise to a normal force which initially increases moderately with increasing excess and increases more pronouncedly in the case of a greater excess. In this way, it is possible to achieve a sufficient sealing effect and, at the same time, compensate for any possible excess in the valve piston 50 and reduce the friction between the valve piston 50 and the valve housing 32.

The sealing lip 65 is formed in one piece with the valve piston 50, and so the latter can be produced in a single injection molding process with the sealing lip 65 molded on. Otherwise, the valve piston 50 is designed in accordance with the embodiments described above.

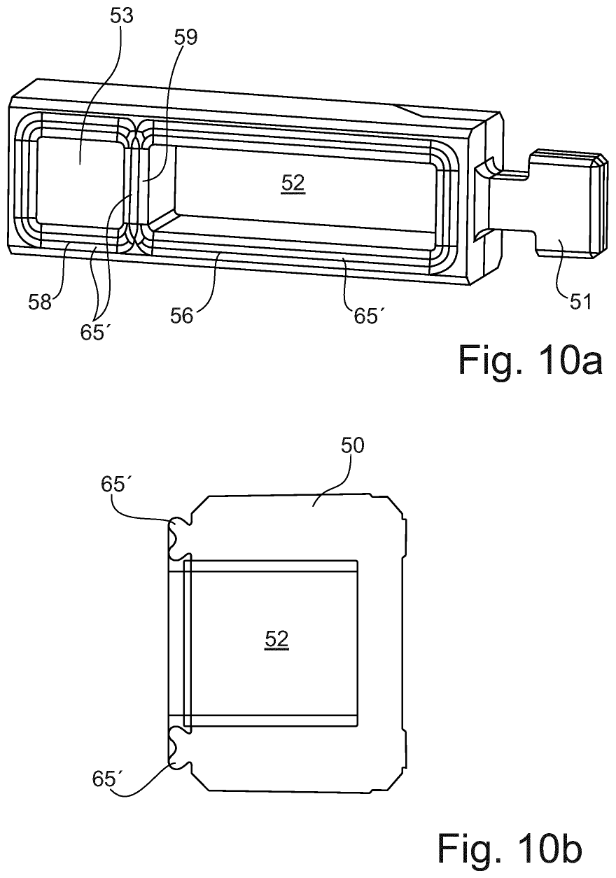

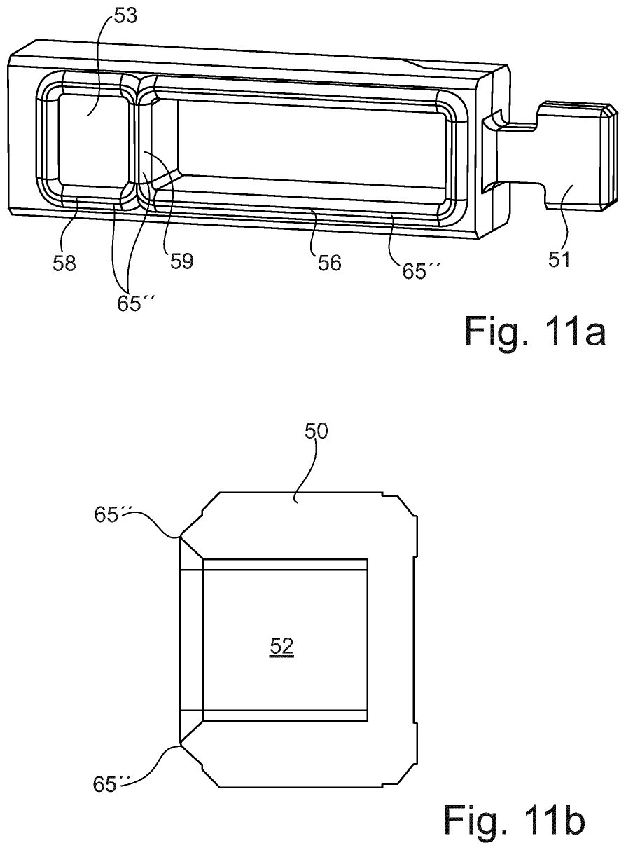

FIGS. 10a and 10b and respectively 11a and 11b show a fifth and respectively a sixth embodiment of the valve piston 50. In this case, the first seal 56, the second seal 58 and the common portion 59 of the first and the second seal 56, 58 are formed by a sealing lip 65′ or 65″, which is V- or X-shaped in cross section (FIGS. 10a and 10b) or wedge-shaped or triangular (FIGS. 11a and 11b). This creates a double or single line-shaped sealing contact, wherein, in the case of an excess in the valve piston 50, the force with which the seal is pressed against a sealing surface likewise increases moderately at the outset and then increases more strongly in the case of a larger excess. The sealing lip 65′ or 65″ is likewise designed in one piece with the valve piston 50. Otherwise, the valve piston 50 is designed as described above.

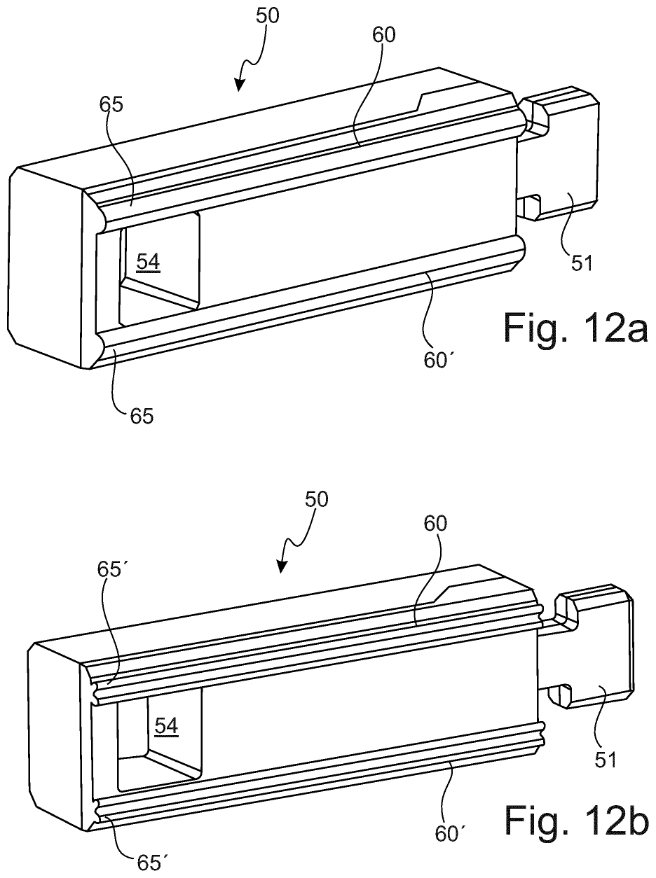

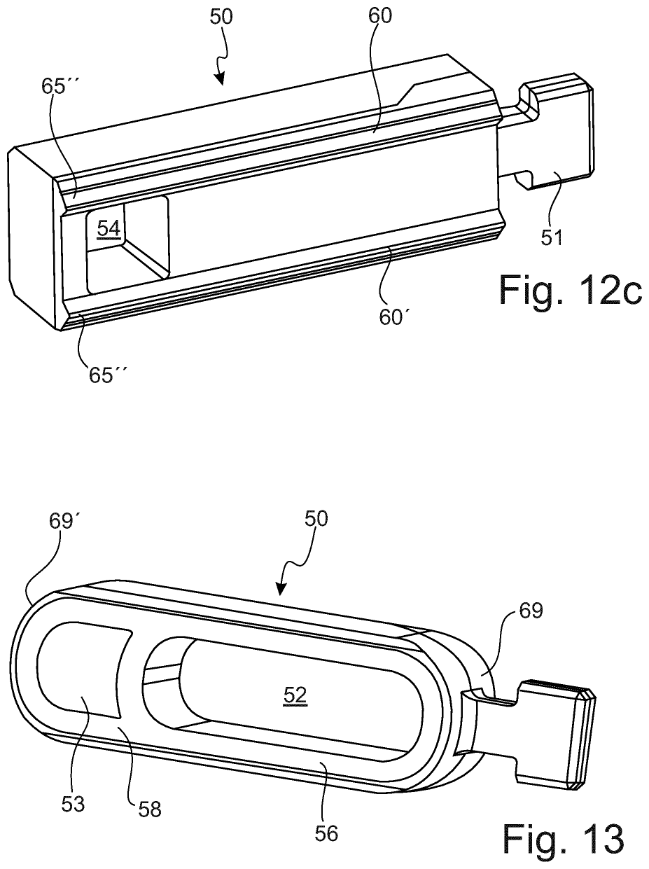

In the above-described embodiments, the counterholder is formed in each case by two parallel, strip-shaped counterholder elements 60, 60′, each of which is designed as a flat elevation 57 (indicated in FIG. 9b by way of example). Alternatively, the counterholder elements 60, 60′ can for example have a semicircular (see FIG. 12a), a V- or X-shaped (see FIG. 12b), or a wedge-shaped or triangular (see FIG. 12c) cross-sectional profile. In this way, as explained above with reference to FIGS. 9a to 11b, a possible excess of the valve piston 50 can be compensated for, the sealing effect of the seals 56, 58 can be improved, and the friction when actuating the valve 30 can be minimized. In particular, the counterholder elements 60, 60′ can each be designed in a manner corresponding to the first and the second seal 56, 58 and the common portion 59 of the first and the second seal 56, 58. The respective counterholder elements 60, 60′ are likewise designed in one piece with the valve piston 50.

As depicted in FIG. 13, the valve piston 50 can have rounded-off end faces 69, 69′ according to a seventh embodiment, which can otherwise be designed in accordance with the above-described embodiments. In particular, the end faces 69, 69′, as seen in longitudinal section, can be designed to be approximately semicircular. A more direct flow of force can be achieved during a displacement of the valve piston 50 by means of the connecting portion 51, and deformation of the valve piston 50 can be reduced. In order to ensure an approximately uniform wall thickness, the recess 52 and the further recess 54, not visible here, also have a correspondingly rounded-off design, and the shape of the first seal 56, which is arranged on the edge of the recess 52 and surrounds the latter all round, and also the shape of the second seal 58, which surrounds the adjoining closure region 53 all round, are adapted accordingly.

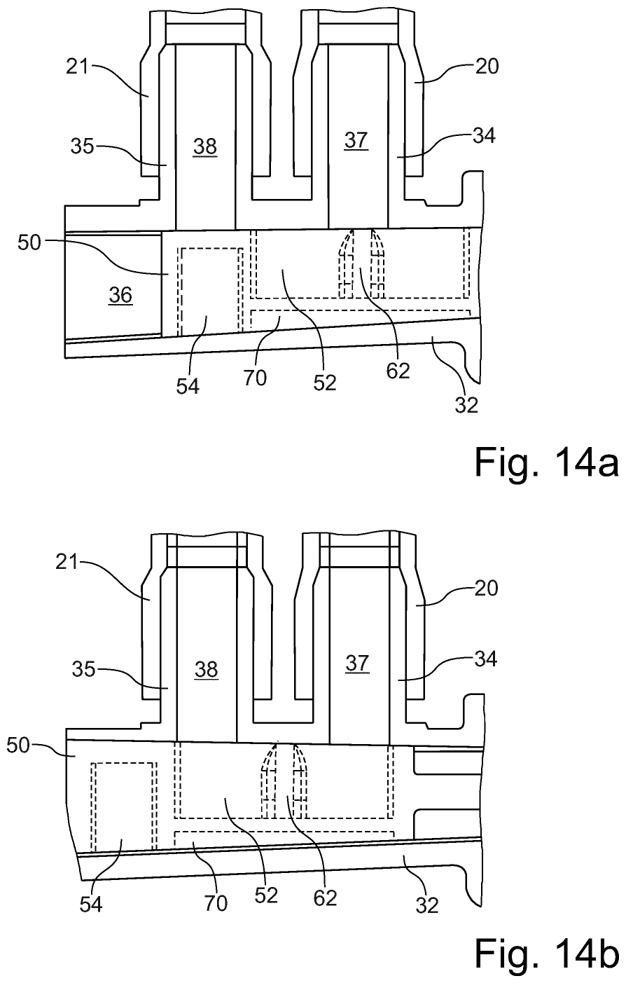

FIGS. 14a and 14b, corresponding to FIGS. 5a and 5c, show a further embodiment of the valve 30 according to the disclosure in the closed position (FIG. 14a) and in the open position (FIG. 14b), respectively. This embodiment differs from the above-described embodiments in that the cavity 36 of the valve housing 32, in which the valve piston 50 is guided, is designed in such a wedge shape that the valve piston 50 abuts on both sides in the closed position (FIG. 14a) and is therefore pressed against the flat sealing surface of the valve housing 32 surrounding the mouths of the outlets 37, 38, while play remains in the open position (FIG. 14b). In the process, there is an adjustment range of the valve piston 50 in which both the seals 56, 58 and the counterholder elements 60, 60′ are in contact with the valve housing 32 at the same time, on account of the deformability of the seals 56, 58 and the counterholder elements 60, 60′. This is advantageous in that increased friction between the valve piston 50 and the valve housing 32 occurs only in this short adjustment range. When used as a suction valve, applied negative pressure in this case can pull the valve piston 50 against the sealing surface of the valve housing 32 even in the open position.

In order to obtain a uniform wall thickness and a sufficiently high stiffness of the valve piston 50, the latter can have a wedge-shaped recess 70 on its side opposite the outlets 37, 38 and, as indicated by way of example in FIGS. 14a and 14b, a reinforcing rib 62 in the cuboid cavity 52. Otherwise, the valve 30 according to the exemplary embodiment shown in FIGS. 14a and 14b is designed as previously described.

For the sake of clarity, not all reference signs are shown in all of the figures. Reference signs not explained in the context of one figure have the same meaning as in the other figures.

1. A valve, in particular a suction valve, for a medical instrument, the valve comprising:
a valve housing with a cavity which extends in a longitudinal direction and into which, on a longitudinal side of the cavity, a first outlet and a second outlet open in a manner offset from one another in the longitudinal direction; and
a valve piston which is displaceable in the cavity in the longitudinal direction between an open position and a closed position of the valve and which has a recess via which, in the open position, the first outlet and the second outlet are fluidically connected to one another, wherein the valve piston has a first seal which surrounds the recess all round.

2. The valve as claimed in claim 1, wherein the valve piston has a closure region offset in the longitudinal direction from the recess, with, in the closed position, the first outlet being fluidically connected to the recess and the second outlet being sealed by the closure region and with the valve piston having a second seal which surrounds the closure region all round.

3. The valve as claimed in claim 1, wherein a sealing surface of the valve housing, with which the first seal interacts, is a substantially flat surface.

4. The valve as claimed in claim 1, wherein a cross section of the cavity is substantially rectangular.

5. The valve as claimed in claim 1, wherein the cavity is designed to narrow in a wedge shape toward the closed position.

6. The valve as claimed in claim 1, wherein the first seal is formed in one piece with the valve piston.

7. The valve as claimed in claim 2, wherein the second seal is formed in one piece with the valve piston.

8. The valve as claimed in claim 1, wherein the first seal is designed as a sealing lip, which has a semicircular, a V-shaped, or a wedge-shaped cross-sectional profile, or as a surface seal.

9. The valve as claimed in claim 2, wherein the second seal is designed as a sealing lip, which has a semicircular, a V-shaped, or a wedge-shaped cross-sectional profile, or as a surface seal.

10. The valve as claimed in claim 2, wherein the second seal is designed with the same cross-sectional profile as the first seal.

11. The valve as claimed in claim 1, further including a counterholder, the counterholder arranged on an opposite side of the valve piston to the first seal.

12. The valve as claimed in claim 2, wherein the counterholder is arranged opposite to the first seal and the second seal.

13. The valve as claimed in claim 11, wherein the counterholder comprises at least one counterholder element which has a semicircular, a V-shaped, or a wedge-shaped cross-sectional profile or is designed as a flat elevation.

14. The valve as claimed in claim 11, wherein the counterholder is formed in one piece with the valve piston.

15. The valve as claimed in claim 1, wherein the valve piston has a substantially uniform wall thickness.

16. The valve as claimed in claim 1, wherein the valve piston has one or more reinforcing ribs.

17. The valve as claimed in any of the preceding claims, wherein at least one end face of the valve piston has a rounded-off form.

18. A medical instrument, in particular a medical endoscope or medical endoscopic instrument, having a valve as claimed in claim 1.
